(19) United States
(12) Patent Application Publication
(10) Pub. No.: US 2024/0389869 A1
(43) Pub. Date: Nov. 28, 2024

(54) MEASUREMENT DEVICES AND METHODS OF USE THEREOF

(71) Applicant: Bard Peripheral Vascular, Inc., Franklin Lakes, NJ (US)

(72) Inventors: Aaron Blank, Chandler, AZ (US); Matthew Cartwright, Chandler, AZ (US); Summer L. Ford, Queen Creek, AZ (US); Ryan P. Giudice, Gilbert, AZ (US)

(73) Assignee: Bard Peripheral Vascular, Inc., Franklin Lakes, NJ (US)

(21) Appl. No.: 18/685,673

(22) PCT Filed: Sep. 30, 2021
(86) PCT No.: PCT/US2021/052926

§ 371 Date: Feb. 22, 2024

(51) Int. Cl. A61B 5/0215; A61B 5/00

(52) U.S. Cl. CPC A61B 5/02152; A61B 5/6852

(57) ABSTRACT

Embodiments herein are directed to a measurement device. The measurement device includes a processing device, a catheter, and a tip portion. The tip portion is disposed at a distal end of the catheter. The tip portion receives the distal end of the catheter. A diaphragm film assembly is coupled to the tip portion at an opposite end that receives the distal end of the catheter. The diaphragm film assembly is configured to flexibly move between a plurality of positions based on an applied pressure that displaces the diaphragm film assembly. A sensor assembly communicatively coupled to the processing device, the sensor assembly is configured to measure the applied pressure displacing the diaphragm film assembly and the processing device determines a concentration of ammonia currently in a fluid at the tip portion.

### TECHNICAL FIELD

The present specification generally relates to measuring pressure and ammonia concentration in a fluid and, more specifically, to a combination measurement device that measures pressure and ammonia concentration in the fluid.

### BACKGROUND

Portal hypertension is an abnormally high blood pressure in a portal vein usually caused by liver cirrhosis. A Transjugular Intrahepatic Portosystemic Shunt (TIPS) placement is the current clinical standard for treatment of portal hypertension. This procedure creates a bypass between a portal and hepatic veins, bypassing the liver, to decompress the portal vein. The target pressure gradient to confirm portal decompression is approximately 5-10 mmHg. The current clinical standard for measuring pressure within a TIPS procedure is by use of a manometer. The manometer provides a +/−2 mmHg resolution for the physician. Furthermore, if portal hypertension is over-relieved and more unfiltered blood than required is being bypassed past the liver, the patient is at an increased risk for developing Hepatic Encephalopathy. Under normal function, the liver converts ammonia into urea and excretes the waste product from the body via urine. However; following a TIPS procedure, ammonia instead gets bypassed from the portal vein through the hepatic vein back into the bloodstream. Certain concentrations of ammonia returning back to the blood stream and not exiting the body as a waste-product may be considered toxic to the brain, and that concentration is generally believed to play a role in the onset of Type-B Hepatic Encephalopathy.

As such, physicians measure indirect pressure measurements pre-procedure, during the procedure, and post-procedure. The manometer typically requires the use of a wedge catheter to perform the measurements, which may require a multitude of steps and required components and only provides indirect measurements that are subjective based on the user's ability to utilize wedge catheter techniques. If an error occurs on the high end of the range, an unsuccessful portal decompression may result such that the physician may not dilate the TIPS stent graft to a large enough diameter and the patient remains with portal hypertension. If an error occurs on the low end of the target range, the physician runs the risk of over-dilating the TIPS stent graft, bypassing an excess of unfiltered blood passed the liver and increasing the patient's risk of developing Hepatic Encephalopathy.

Accordingly, a need exists for a more accurate measuring system that requires less components, less steps, and one device for direct and indirect measurements.

### SUMMARY

In one aspect, a measurement device is provided. The measurement device includes a processing device, a catheter, and a tip portion. The tip portion is disposed at a distal end of the catheter. The tip portion receives the distal end of the catheter. A diaphragm film assembly is coupled to the tip portion at an opposite end that receives the distal end of the catheter. The diaphragm film assembly is configured to flexibly move between a plurality of positions based on an applied pressure that displaces the diaphragm film assembly. A sensor assembly communicatively coupled to the processing device, the sensor assembly is configured to measure the applied pressure displacing the diaphragm film assembly and the processing device determines a concentration of ammonia currently in a fluid at the tip portion.

In another aspect, a combination measurement device is provided. The combination measurement device includes a processing device, a handle, a catheter, and a tip portion. The catheter extends from the handle. The tip portion is disposed at a distal end of the catheter such that the tip portion is positioned opposite of the handle. The tip portion includes a first end portion, an opposite second end portion, and a cavity extending therebetween. The first end portion receives the distal end of the catheter. The sensor assembly is positioned within the cavity of the tip portion. The diaphragm film assembly is coupled to the tip portion and communicatively coupled to the sensor assembly and to the processing device. The diaphragm film assembly is configured to flexibly move between a plurality of positions based on an applied pressure that displaces the diaphragm film assembly. The sensor assembly is configured to measure the applied pressure displacing the diaphragm film assembly and the processing device is configured to determine a concentration of ammonia currently in a fluid at the tip portion.

In yet another aspect, a method for determining a pressure and a concentration of ammonia within a fluid is provided. The method includes detecting, with a sensor assembly, an amount of a deflection currently occurring in a diaphragm film assembly positioned at a tip portion of an elongated member, the diaphragm film assembly is communicatively coupled to the sensor assembly, detecting, with a processing device, a change in a resistance of diaphragm film assembly, and displaying, on a display portion communicatively coupled to the sensor assembly and to the processing device, the amount of the deflection currently occurring and the change in the resistance of diaphragm film assembly. The change in the resistance of diaphragm film assembly is indicative of a change in the concentration of ammonia and an amount of deflection currently occurring in the diaphragm film assembly is indicative of the pressure.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 schematically depicts a combination measuring device, according to one or more embodiments shown and described herein;

FIG. 2 schematically depicts an isolated cross sectional view of a tip portion of the combination measuring device of FIG. 1 taken from the line 2-2, according to one or more embodiments shown and described herein;

FIG. 3A schematically depicts an isolated side view of a tip portion of the combination measuring device of FIG. 1, according to one or more embodiments shown and described herein;

FIG. 3B schematically depicts an isolated perspective view of a tip portion of the combination measuring device of FIG. 1, according to one or more embodiments shown and described herein;

FIG. 4A schematically depicts an isolated cross sectional view of a diaphragm assembly of the tip portion of FIG. 3B taken from the line 4-4 in a static state, according to one or more embodiments shown and described herein;

FIG. 4B schematically depicts an isolated cross sectional view of a diaphragm assembly of the tip portion of FIG. 3B taken from the line 4-4 in a displaced state, according to one or more embodiments shown and described herein;

FIG. 5A schematically depicts an isolated view of a display portion of the combination measuring device of FIG. 1, the display portion illustrating a measured pressure, according to one or more embodiments shown and described herein;

FIG. 5B schematically depicts an isolated view of a display portion of the combination measuring device of FIG. 1, the display portion illustrating an ammonia concentration, according to one or more embodiments shown and described herein;

FIG. 6 schematically depicts an electrical diagram of the combination measuring device of FIG. 1, according to one or more embodiments shown and described herein;

FIG. 7 schematically depicts an isolated view of a second aspect of the diaphragm assembly of FIG. 2, according to one or more embodiments shown and described herein;

FIG. 8 schematically depicts an isolated view of a second aspect of the catheter and the tip portion of FIG. 2, according to one or more embodiments shown and described herein;

FIG. 9 depicts a flowchart of an illustrative method for determining a pressure and a concentration of ammonia within a fluid, according to one or more embodiments shown and described herein;

FIG. 10A schematically depicts the combination measuring device of FIG. 1 positioned within a hepatic vein pre-Transjugular Intrahepatic Portosystemic Shunt placement, according to one or more embodiments shown and described herein;

FIG. 10B schematically depicts the combination measuring device of FIG. 1 positioned within a portal vein pre-Transjugular Intrahepatic Portosystemic Shunt placement, according to one or more embodiments shown and described herein; and

FIG. 10C schematically depicts the combination measuring device of FIG. 1 positioned within a portal vein post-Transjugular Intrahepatic Portosystemic Shunt placement, according to one or more embodiments shown and described herein.

### DETAILED DESCRIPTION

Embodiments described herein generally relate to a combination measurement device that may be utilized in conjunction with a covered stent/shunt during a Transjugular Intrahepatic Portosystemic Shunt (“TIPS”) procedure. The combination measurement device includes a catheter, a tip portion, a sensor assembly, and a diaphragm film assembly. The tip portion may disposed at a distal end of the catheter such that the tip portion of the device may be inserted via a minimally invasive technique to an anatomy location of interest and may be used in a manner to obtain direct pressure measurements at the hepatic and portal veins. The diaphragm film assembly is configured to flexibly move between a plurality of positions based on an applied pressure caused from a fluid present at the tip portion. The fluid displaces the diaphragm film assembly. The sensor assembly measures the applied pressure displacing the diaphragm film assembly by determining a change in capacitance. Additionally, a concentration of ammonia currently in the fluid at the tip portion may be determined by a processing device based on a change in resistance, for example. As such, the change in capacitance correlates to a pressure applied against the tip portion and the change in resistance correlates to an associated concentration of ammonia that bypasses the liver into the hepatic veins.

As such, the embodiments described herein produce desirable results over existing solutions, such as the ability to obtain instantaneous, direct, and accurate pressure measurements and to monitor real-time concentrations of nitrogenous compounds. As such, physicians may anticipate the onset of hepatic encephalopathy (“HE”) such that adjustments to shunt dilation parameters may be performed to successfully reach portal decompression while reducing the risk of HE onset.

Various embodiments of combination measurement devices and methods of use thereof are described in detail herein.

As used herein, the term “communicatively coupled” may mean that coupled components are capable of exchanging data signals with one another such as, for example, electrical signals via conductive medium or a non-conductive medium, though networks such as via Wi-Fi, Bluetooth, and the like, electromagnetic signals via air, optical signals via optical waveguides, and the like.

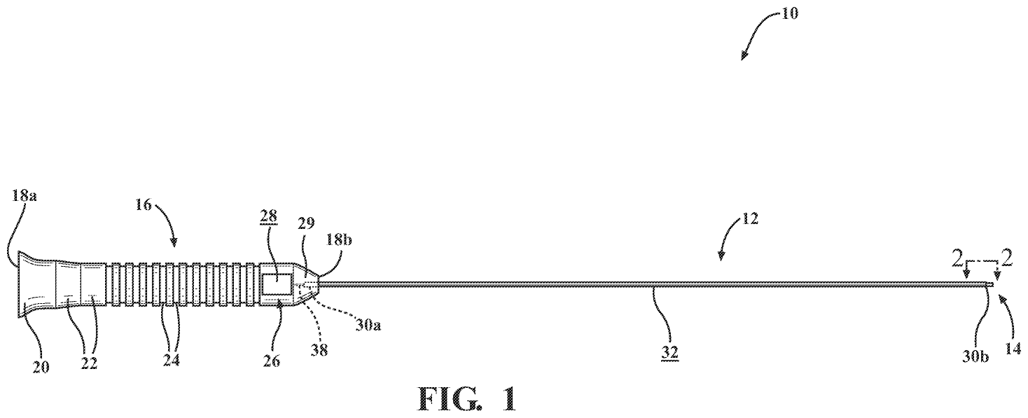

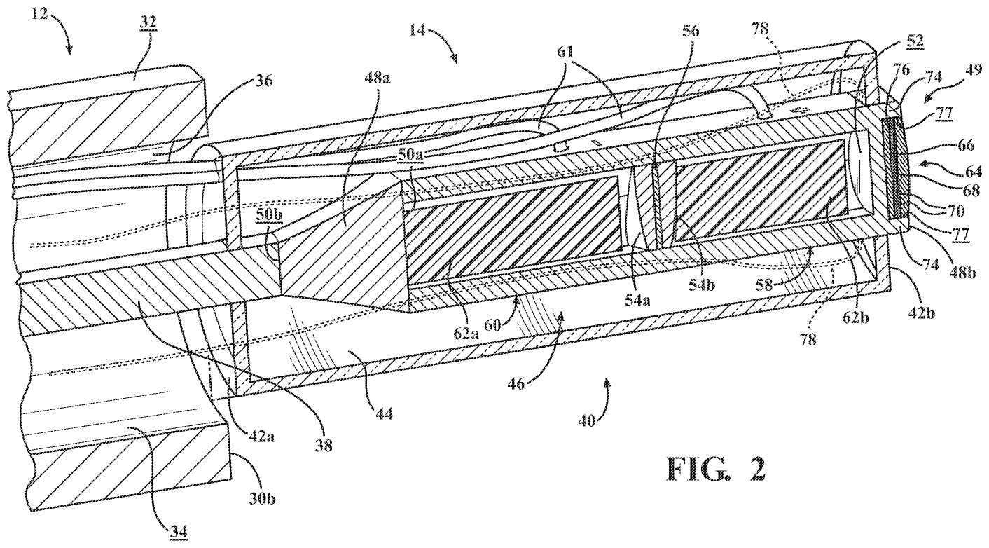

Referring initially to FIGS. 1-2, a combination measurement device 10 is schematically depicted. The combination measurement device 10 may be utilized in conjunction with a covered stent/shunt during a TIPS procedure, though other uses are contemplated and possible. The combination measurement device 10 generally includes a catheter 12 and a tip portion 14. The combination measurement device 10 may further include a handle 16. The handle 16 may include a handle first end 18a and an opposite handle second end 18b that define a length of the handle 16 therebetween. In some embodiments, the handle first end 18a and the handle second end 18b may be generally cylindrically shaped so to define a diameter that extends along the length. As such, in some embodiments, the handle 16 may be used to move, navigate, and/or manipulate the catheter 12 and the tip portion 14 of the combination measurement device 10, as discussed in greater detail herein. The handle 16 may include a flared portion 20 at or adjacent to the handle first end 18a. In some embodiments, the flared portion 20 may provide additional support for the user to move, navigate or manipulate the catheter 12 and tip portion 14 during a TIPS procedure, as discussed in greater detail herein.

In some embodiments, the handle 16 may further include one or more tapered portions 22 that taper the handle 16 in a direction away from the handle first end 18a and towards the handle second end 18b. Further, the handle 16 may include a plurality of indentions 24. The plurality of indentions 24 may extend circumferentially around the handle 16 between the one or more tapered portions 22 and the handle second end 18b. In some embodiments, the plurality of indentions 24 may be uniformly spaced from one another. In other embodiments, some or all of the plurality of indentions 24 are non-uniformly, or irregularly, spaced from one another. It should be appreciated that the plurality of indentions 24 may provide the user with additional support and/or a textured grip for the user to navigate the catheter 12 and the tip portion 14 during the TIPS procedure, as discussed in greater detail herein.

In some embodiments, the handle 16 may further include a display device 26. The display device 26 may include a display 28 that outputs measurement data sensed by the combination measurement device 10, as discussed in greater detail herein. In some embodiments, the display 28 of the display device 26 may display measurement data related to a measured pressure determined by the combination measurement device 10. In other embodiments, the display 28 of the display device 26 may display measurement data related to a measured ammonia concentration, as discussed in greater detail herein. In other embodiments, the display device 26 may be configured to, or capable of, switching display outputs between the measurement data related to the measured pressure and the measurement data related to the measured ammonia concentration, as discussed in greater detail herein. Further, in other embodiments, the display device 26 may be configured to, or capable of, displaying outputs of both the measurement data related to the measured pressure and the measurement data related to the measured ammonia concentration simultaneously. In some embodiments, the display device 26 may be a touch screen or there may be other user input devices mounted to the handle 16 (e.g., buttons, toggles, switches, etc.) to allow a user to navigate a menu and/or information displayed on the display device 26.

In some embodiments, between the display device 26 and the handle second end 18b, the handle 16 may further include a second one or more tapered portions 29 that tapers the handle 16 in a direction away from the display device 26 and towards the handle second end 18b, thereby further decreasing a cross-section of the handle 16.

The handle 16 may be formed of any material to be handled by a user, such as a surgical grade polymer material. For example, the handle 16 may be polysulfone, a polypropylene, a polycarbonate, and/or the like. In other embodiments, the handle 16 may be a surgical stainless steel, surgical grade plastics, or the like, usable in biomedical applications). For example, the handle 16 may be an austenitic SAE 316 stainless steel, a martensitic SAE 440 stainless steel, a SAE 420 stainless steel, a 17-4 stainless steel, and/or the like. In other embodiments, the handle 16 may include a textured surface. It should be appreciated that the handle 16 may be formed from a combination of surgical materials.

Still referring to FIGS. 1-2, the catheter 12 includes a proximal end 30a and an opposite distal end 30b that define a length of the catheter 12. Further, in some embodiments, the distal end 30b and the proximal end 30a are cylindrical in shape to define a diameter along the length of the catheter 12. For example, the catheter 12 may be tubular such that the catheter 12 defines an outer surface 32 and an inner surface 34. The inner surface 34 may define a lumen 36 of the catheter 12, which may extend the length of the catheter 12. In some embodiments, the catheter 12 have differential cross-section shapes other than circular, for example, rectangular, hexagonal, octagonal, and/or the like. Further, in some embodiments, the catheter 12 may have a uniform shape between the proximal and distal ends 30a, 30b. In other embodiments, the catheter 12 may be non-uniform or have irregular portions between the proximate and distal ends 30a, 30b.

Positioned within the lumen 36 of the catheter 12 may be a guide wire 38. In some embodiments, the guide wire 38 may extend beyond the proximal and distal ends 30a, 30b. The guide wire 38 may have any cross-section shape, for example circular, rectangular, hexagonal, octagonal, cylindrical, and/or the like. Further, in some embodiments, the guide wire 38 may have a uniform shape between the proximal and distal ends 30a, 30b. In other embodiments, the guide wire 38 may be non-uniform or have irregular portions between the proximal and distal ends 30a, 30b.

Still referring to FIGS. 1-2, the proximal end 30a of the catheter 12 may be received by the handle second end 18b of the handle 16 to couple the catheter 12 to the handle 16. For example, the proximal end 30a of the catheter 12 may be received within a port formed within the handle 16 and secured thereto via, for example, in a snap fit configuration. In other embodiments, the proximal end 30a of the catheter 12 may be received within a port formed within the handle second end 18b of the handle 16 and secured thereto via one or more fasteners, welding, adhesives, interlocking members, hook and loop connection, and/or the like. In embodiments, the catheter 12 and the handle 16 may be co-axially aligned. That is, a centerline of the catheter 12 and the handle 16 may be coincident with one another. However, in other embodiments, the catheter 12 and the handle 16 may not be coaxial.

The catheter 12 and/or the guide wire 38 may be a surgical grade material such as a surgical stainless steel used in biomedical applications. For example, the catheter 12 and/or the guide wire 38 may be an austenitic SAE 316 stainless steel, a martensitic SAE 440 stainless steel, a SAE 420 stainless steel, a 17-4 stainless steel, and/or the like. In other embodiments, the catheter 12 and/or the guide wire 38 may be a surgical grade polymer material, surgical grade plastics, or the like, usable in biomedical applications. For example, the catheter 12 and/or the guide wire 38 may be polysulfone, a polypropylene, a polycarbonate, and/or the like. It should be appreciated that the catheter 12 and/or the guide wire 38 may be formed from a combination of surgical materials.

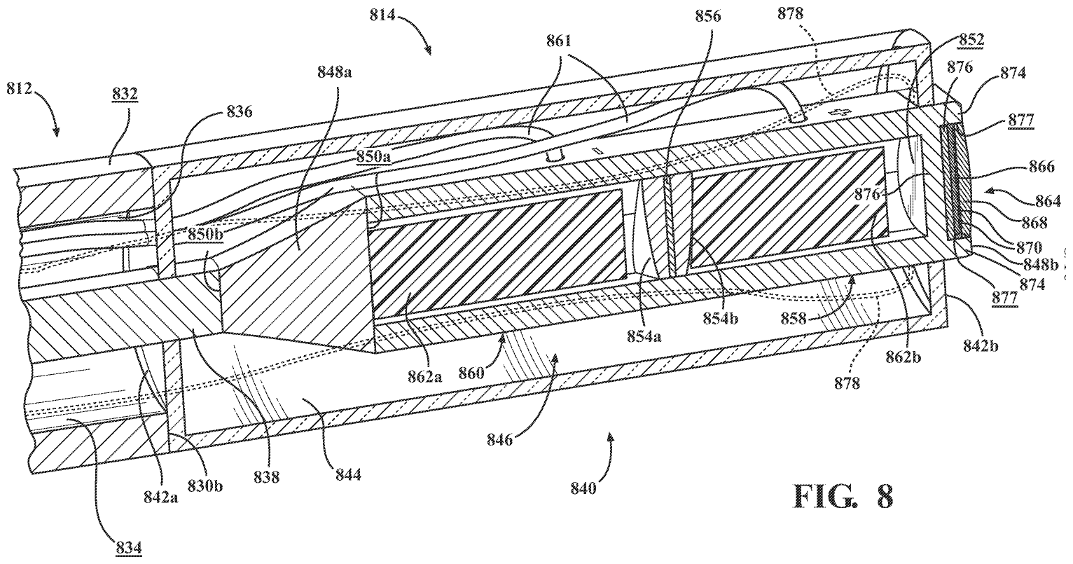

Now referring to FIGS. 1-3B, the tip portion 14 extends from the distal end 30b of the catheter 12. In some embodiments, the tip portion 14 is a separate component that may be movable with respect to the distal end 30b of the catheter 12. In some embodiments, the tip portion 14 may be coupled to the distal end 30b of the catheter 12 (e.g., via one or more fasteners, welding, adhesives, interlocking members, hook and loop connection, snap fit connection, and/or the like). In other embodiments, the tip portion 14 is integrally formed with the catheter 12, as illustrated best in FIG. 8. The tip portion 14 may generally include a housing 40. The housing 40 has a first end 42a and an opposite second end 42b. A length of the housing 40 extends between the first end 42a and the second end 42b. Further, in some embodiments, the first end 42a and the second end 42b may be cylindrical in shape. As such, the housing 40 has a diameter along the length of the tip portion 14 between the first end 42a and the second end 42b. It should be appreciated that, in some embodiments, the diameter of the housing 40 may be smaller, equal to, or larger than the diameter of the catheter 12. Further, in other embodiments, the housing 40 may have different or changing diameters along its length such that a diameter of the housing 40 is non-uniform along its length. It is noted that while the cross-section of the housing may be circular, in other embodiments the cross-section of the housing may be rectangular, hexagonal, octagonal, or any regular or irregular, polygonal or non-polygonal shape.

The housing 40 may define a cavity 44 that extends between the first and second ends 42a, 42b such that the cavity 44 is accessible from or through both the first and second ends 42a, 42b. A sensor assembly 46 may be disposed within the cavity 44 or otherwise mounted to the housing 40. For example, the sensor assembly 46 may include a mating end 48a and a sensor end 48b that define a length of the sensor assembly 46 therebetween. The mating end 48a may be tapered from an inner mating surface 50a and terminating at an outer mating surface 50b. The sensor end 48b includes a pocket portion 49. The pocket portion 49 includes a pair of legs 74. The pair of legs 74 of the pocket portion 49 may be spaced apart and generally extend in a direction perpendicular or orthogonal to an inner sensor surface 52 of a diaphragm film assembly 64, as discussed in greater detail herein. Each of the pair of legs 74 include an inner surface 77.

Portions of the sensor end 48b and/or the outer sensor surface 52b may extend beyond the second end 42b of the housing 40 of the tip portion 14, as depicted in FIG. 2. In other embodiments, it is contemplated that the sensor end 48b may be flush with or recessed relative to second end 42B of the housing 40.

Still referring to FIG. 2, in some embodiments, the guide wire 38 may be a monolithic structure extending from the catheter 12 through the tip portion 14. In other embodiments, the outer mating surface 50b of the mating end 48a of the sensor assembly 46 may couple to an end of the guide wire 38 (for example, via epoxy, adhesive, weld, and/or the like). For example, the mating end 48a may terminate at the same diameter at the guide wire 38 such that the portion of the guide wire 38 that extends beyond the distal end 30b of the catheter 12 is coupled to the outer mating surface 50b of the mating end 48a. That is, the portion of the guide wire 38 that extends beyond the distal end 30b of the catheter 12 may be coupled to or connected to the outer mating surface 50b. In some embodiments, the coupling or connecting of the guide wire 38 to the outer mating surface 50b may be via a fastener such as a screw, rivet, bolt and nut, and the like. In other embodiments, the coupling or connecting of the guide wire 38 to the outer mating surface 50b may be via a solder, weld, adhesive, epoxy, and/or the like. In another embodiment, guide wire 38 may be housed in an individual lumen in the wall 32 such that the sensor assembly 46 slidably moves along the path created by the guide wire 38. In some embodiments, the tip portion 14 and the catheter 12 may be co-axially aligned. That is, a centerlines of the tip portion 14 and the catheter 12 may be coincident with one another. In other embodiments, the tip portion 14 and the catheter 12 may not be coaxial.

Still referring to FIG. 2, in some embodiments, the tip portion 14 is slidably movable with respect to the catheter 12 via the guide wire 38 between a deployed position, as shown in FIG. 2 and a home position, as illustrated by the dashed-dot-dashed lines in FIG. 2. In the home position, the first end 42a of the tip portion 14 abuts a portion of the distal end 30b of the catheter 12. In the deployed position, the first end 42a of the tip portion 14 is spaced apart from the distal end 30b of the catheter 12. It should be appreciated that, in the deployed position, the first end 42a of the tip portion 14 may be spaced apart from the distal end 30b of the catheter 12 a plurality of different distances and is only limited by the length of the guide wire 38. As such, the deployed position allows for the tip portion 14 to extend away from the catheter 12 to gather measurement data, as discussed in greater detail herein.

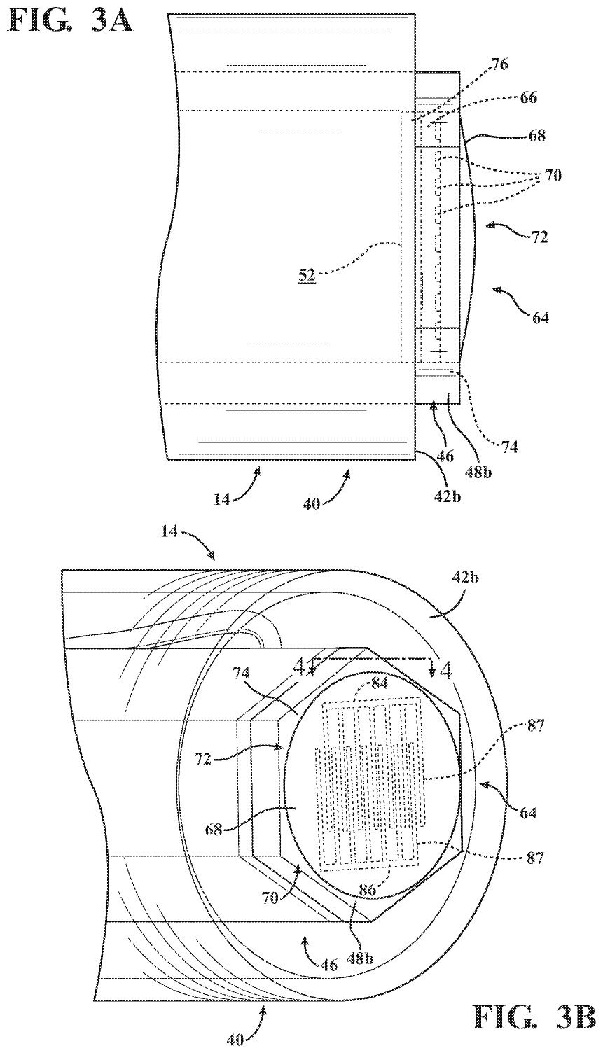

Now referring to FIGS. 2-3B, the sensor assembly 46 may include a capacitive micro-electro-mechanical systems type sensor configured to output a plurality of signals, as discussed in greater detail herein. For example, the sensor assembly 46 may be a micro-pressure board mount pressure sensor, for example an MPR series micro-pressure board mount pressure sensors, such as manufacturer No. MPRSS0001PG00001C.

For example, the sensor assembly 46 may include a pair of capacitance plates 54a, 54b that are spaced apart by a dielectric material 56. The dielectric material 56 may be a solid, a liquid and/or a gas. For example, and without limitation, the solid dielectric material may be ceramic, paper, mica, glass, and the like. The liquid dielectric material may be distilled water, transformer oil, and the like. The gas dielectric material may be nitrogen, dry air, helium, oxides of various metals, and the like. Each of the pair of capacitance plates 54a, 54b may be made from a conductive material such as, without limitation, aluminum, tantalum, silver, or other metals.

The pair of capacitance plates 54a, 54b may be positioned between the mating end 48a and the sensor end 48b. As such, the one of the pair of capacitance plates 54a and the inner mating surface 50a form a first capacitor and the other one of the pair of capacitance plates 54b and an inner sensor surface 52 of the diaphragm film assembly 64 form a second, different capacitor. An insulation material 62a may positioned circumferentially between the capacitance plate 54a and the inner mating surface 50a and an insulation material 62b may be positioned circumferentially between the capacitance plate 54b and the inner sensor surface 52 of the diaphragm film assembly 64. The insulation material 62a, 62b may be arranged in layers and may be any nonconductive material to provide electrical insulating. For example, the insulation material 62a, 62b may be, without limitation, Teflon®, polyethylene, polyimide, polypropylene, polystyrene, and the like.

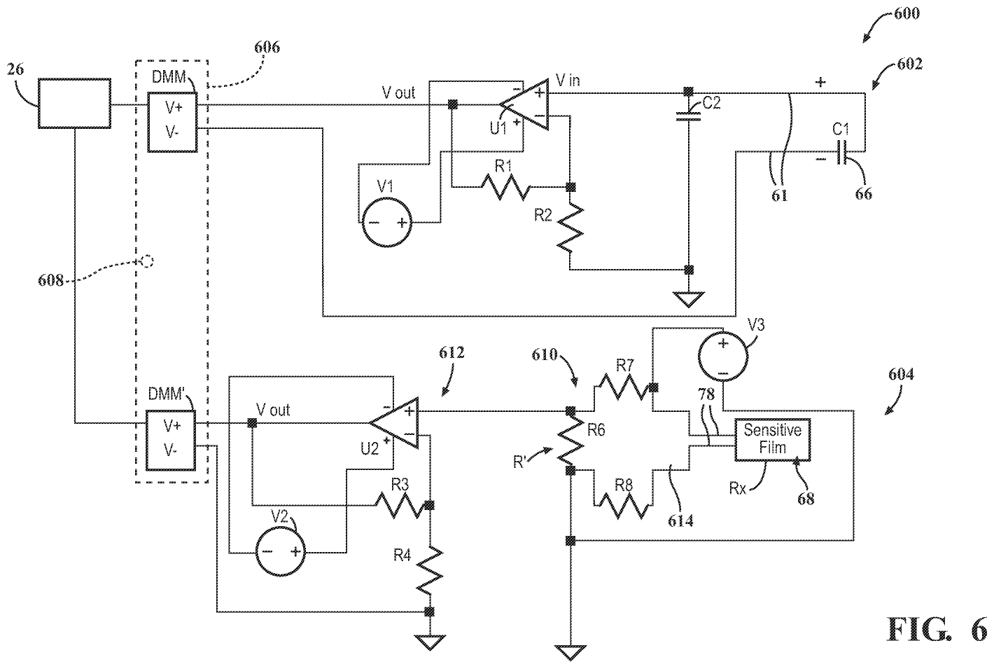

Each of the pair of capacitance plates 54a, 54b may be used to output a capacitance between the respective capacitance plate 54a, 54b and either the corresponding inner mating surface 50a or inner sensor surface 52. The first capacitor may be a sensing capacitor 58 while the second capacitor may be a reference capacitor 60. The sensing capacitor 58 and the reference capacitor 60 of the sensor assembly 46 may each be communicatively coupled to a processing device 606 (FIG. 6) via traditional electrical connection appreciated by those skilled in the art. For example, by a plurality of positive and negative wires connections 61.

As discussed in greater detail herein, as the inner sensor surface 52 of the diaphragm film assembly 64 flexes, displaces, or moves based on a pressure applied at the diaphragm film assembly 64, which changes a capacitance of the sensing capacitor 58. That is, the movement of the inner sensor surface 52 of the diaphragm film assembly 64 changes a capacitance measured between the capacitance plate 54b and the inner sensor surface 52 of the diaphragm film assembly 64 (e.g., the sensing capacitor 58). The change in capacitance value at the sensing capacitor 58 may be compared to a measurement from the reference capacitor 60 to detect a change or ratio in a measurement data by a processing device 606 (FIG. 6).

Now referring to FIGS. 2-4B, the diaphragm film assembly 64 is configured to detect, or sense, a pressure and an ammonia concentration within a fluid at the diaphragm film assembly 64 and may be disposed within or coupled to the pocket portion 49 of the sensor end 48b. In some embodiments, at least a portion of the diaphragm film assembly 64 is positioned within and/or coupled to the pocket portion 49. As such, the diaphragm film assembly 64 may extend outwardly from portions of the sensor assembly 46 and beyond the second end 42b of the housing 40 of the tip portion 14.

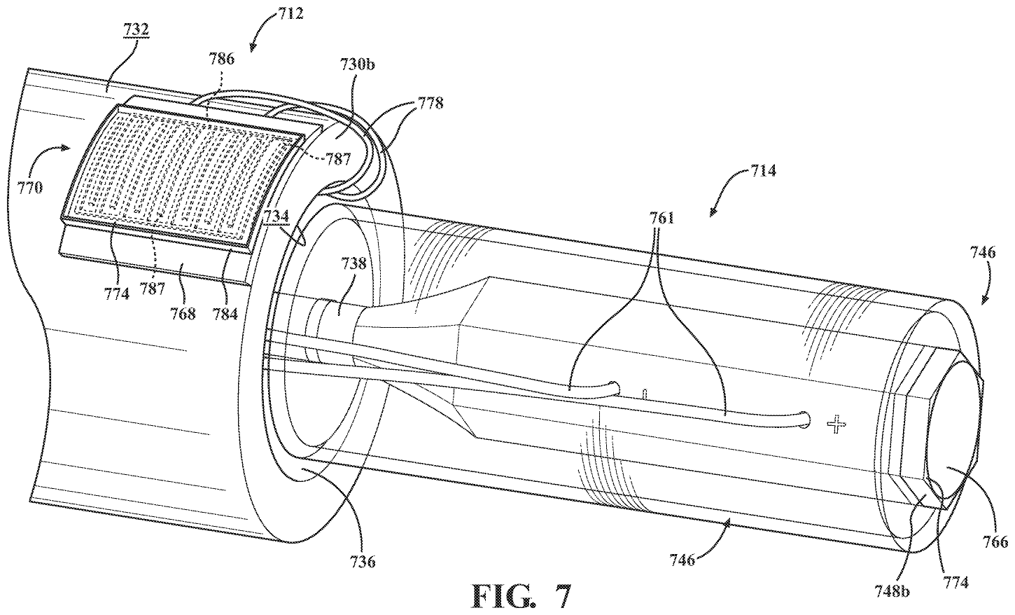

The diaphragm film assembly 64 may be coupled to the inner surface 77 of the pair of legs 74 of the pocket portion 49 via any fastening element such as, but not limited to, epoxy, adhesive, weld, screw, rivet, and/or the like. In other embodiments, the diaphragm film assembly 64, and/or portions thereof, may be coupled to other portions of the combination measurement device 10 such as to the catheter 12, as discussed in greater detail herein with respect to FIG. 7. Further, the diaphragm film assembly 64 is positioned to be spaced apart from the capacitance plate 54b.

In one embodiment, the diaphragm film assembly 64 may include a capacitance layer film member 66 (also referred to herein as the second film layer), a sensitive layer film member 68 (also referred to herein as the first film layer), and a pair of electrodes 70. In these embodiments, the capacitance layer film member 66 and the sensitive layer film member 68 may each be attached or coupled to the pair of legs 74 of the pocket portion 49. As such, the capacitance layer film member 66 and the sensitive layer film member 68 freely move within the pocket portion 49 based on a pressure amount applied, while remaining coupled or attached to the pair of legs 74 of the pocket portion 49, as discussed in greater detail herein. Further, in these embodiments, the capacitance layer film member 66, the pair of electrodes 70, and the sensitive layer film member 68 are stacked in an axial direction such that the pair of electrodes 70 are embedded between the capacitance layer film member 66 and the sensitive layer film member 68, and such that a portion of the capacitance layer film member 66 forms the inner sensor surface 52 of the diaphragm film assembly 64. That is, a portion of the inner surface of the capacitance layer film member 66 closest to the capacitance plate 54b may be the inner sensor surface 52 of the diaphragm film assembly 64. The capacitance plate 54b and the inner sensor surface 52 forms the sensing capacitor 58.

In other embodiments, the diaphragm film assembly 64 further includes a base attachment layer film member 72. In these embodiments, the base attachment layer film member 72 may be attached or coupled to the pair of legs 74 of the pocket portion 49. As such, the base attachment layer film member 72 may form a base for the capacitance layer film member 66 and the sensitive layer film member 68 to rest thereon. In other embodiments, the capacitance layer film member 66 and the sensitive layer film member 68 may be coupled or attached to the base attachment layer film member 72. In these embodiments, as illustrated, the base attachment layer film member 72, capacitance layer film member 66, the pair of electrodes 70, and the sensitive layer film member 68 are stacked in an axial direction such that the pair of electrodes 70 are embedded between the capacitance layer film member 66 and the sensitive layer film member 68, and such that a portion of the base attachment layer film member 72 forms the inner sensor surface 52 of the diaphragm film assembly 64. That is, a portion of the inner surface of the base attachment layer film member 72 closest to capacitance plate 54b forms may be the inner sensor surface 52 of the diaphragm film assembly 64. The capacitance plate 54b and the inner sensor surface 52 forms the sensing capacitor 58.

It should be appreciated that the base attachment layer film member 72 and/or the capacitance layer film member 66 may flex, displace or move in response to a pressure of a fluid applied at the capacitance layer film member 66. This flexing, displacement or moving changes or modifies a capacitance as sensed or measured by the sensing capacitor 58, as discussed in greater detail herein. In embodiments, the flexing, displacement or moving of the inner sensor surface 52 of the diaphragm film assembly 64, with respect to the capacitance plate 54b, may change or modify a current capacitance measured in the sensing capacitor 58.

As such, the capacitance layer film member 66 may remain communicatively coupled to the sensing capacitor 58 regardless of the various mounting embodiments, as discussed in greater detail herein. Further, it should be appreciated the capacitance sensed or measured by the reference capacitor 60 may be indicative of an environment that the sensing assembly 46 is currently being subjected to such that a difference between the environment and the change in capacitance as sensed or measured by the sensing capacitor 58 can be realized.

In some embodiments, portions of the capacitance layer film member 66 and/or the sensitive layer film member 68 may be mounted to the pair of legs 74 via a contact layer such as an adhesive, an epoxy, a liquefied contact metal such as tin, zinc or aluminum, and/or the like. As such, the pair of legs 74 may support a movement of the base attachment layer film member 72, the capacitance layer film member 66 and/or the sensitive layer film member 68, as discussed in greater detail herein.

As illustrated, the base attachment layer film member 72 is positioned closest to the capacitance plate 54b followed by the capacitance layer film member 66 and then the sensitive layer film member 68 with the plurality of electrodes 70 embedded therein. As such, the sensitive layer film member 68 is the most external layer, or is the furthest away from the sensor assembly 46 while the capacitance layer film member 66 is positioned between the sensitive layer film member 68 and the base attachment layer film member 72. As such, it should be appreciated that the different layers of the diaphragm film assembly 64 are positioned in a stacked arrangement.

In some embodiments, the base attachment layer film member 72 may be a polymer material such as, without limitation an aliphatic polyester homopolymer or co-polymer such as PCL, PLA, PGLA or other biocompatible polymers such as chitosan, hyaluronan, or polyurethane with monomers such as PCL, PLA, or a cellulose-based polymer. The base attachment layer film member 72 may also include of a blend of the previously mentioned materials.

In some embodiments, the capacitance layer film member 66 may be a capacitive film material. For example, the capacitive film material may be polyester, polypropylene, polycarbonate and/or the like. In other embodiments, the capacitance layer film member 66 may be other polymer materials such as, without limitation an aliphatic polyester homopolymer or co-polymer such as PCL, PLA, PGLA or other biocompatible polymers such as chitosan, hyaluronan, or polyurethane with monomers such as PCL, PLA, or a cellulose-based polymer. The capacitance layer film member 66 may also include of a blend of the previously mentioned materials.

Still referring to FIGS. 2-4B, in some embodiments, the sensitive layer film member 68 may be a PGA Zinc-Oxide Loaded Polymer material. In other embodiments, the sensitive layer film member 68 may be other polymer materials such as, without limitation an aliphatic polyester homopolymer or co-polymer such as PCL, PLA, PGLA or other biocompatible polymers such as chitosan, hyaluronan, or polyurethane with monomers such as PCL, PLA, or a cellulose-based polymer. The sensitive layer film member 68 may also include of a blend of the previously mentioned materials. It should be understood that the material of the sensitive layer film member 68 may be configured to absorb ammonia such that a resistance in the sensitive layer film member 68 may change based on the amount of ammonia absorbed and this change of resistance may be measured via the sensor assembly 46 and/or via the pair of electrodes 70, as discussed in greater detail herein.

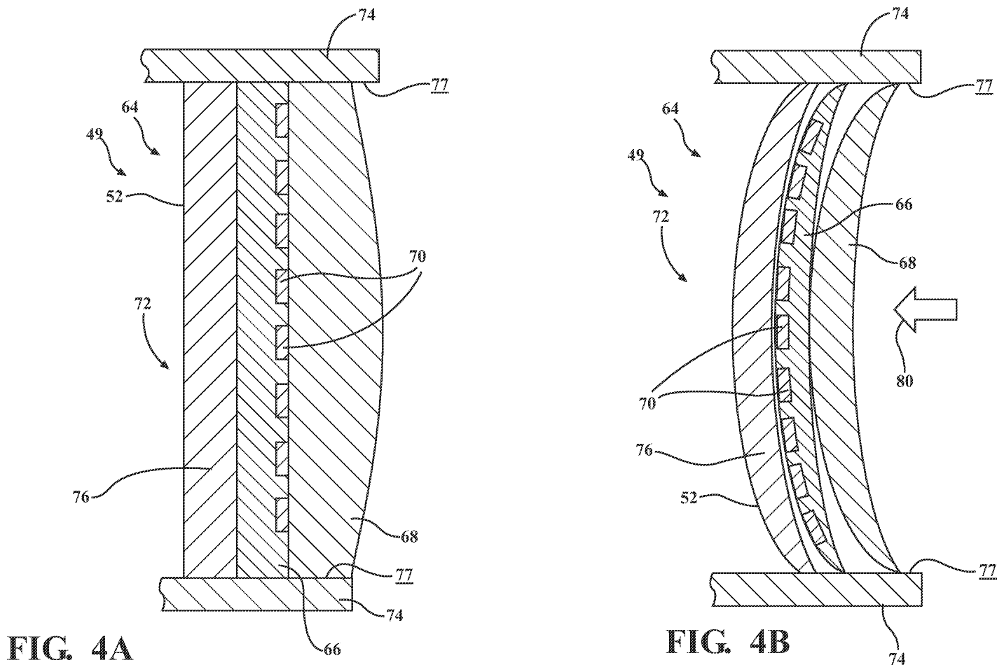

Still referring to FIGS. 3A-4B, the capacitance layer film member 66, the sensitive layer film member 68, and the pair of electrodes 70 may each be positioned or disposed external to the sensor end 48b and the second end 42b of the housing 40 in a static position. It should be understood that the static position may be when there is not a pressure from a fluid, or enough of a pressure exerted onto the capacitance layer film member 66 to move or deflect the capacitance layer film member 66.

Further, the capacitance layer film member 66 and the sensitive layer film member 68 are stacked on the base attachment layer film member 72 in side-by side relationship along an axial direction of the combination measurement device 10. In some embodiments, the pair of electrodes 70 are positioned external to the sensitive layer film member 68. That is, the pair of electrodes 70 extend from the sensitive layer film member 68 in a direction opposite of the capacitance layer film member 66. In other embodiments, the pair of electrodes 70 are positioned or disposed within the capacitance layer film member 66 and/or within the sensitive layer film member 68. Accordingly, the pair of electrodes 70 may be positioned, embedded, or disposed within the capacitance layer film member 66 between the sensitive layer film member 68 and the base attachment layer film member 72 or within the sensitive layer film member 68.

Further, the pair of electrodes 70 may be interdigitated and arranged in two individually addressable comb shaped electrodes 84, 86. As such, the comb shaped electrodes 84, 86 each comprise a plurality of fingers 87 or tangs that are interdigitated with one another. Each of the comb shaped electrodes 84, 86 of the plurality of electrodes 70 are coupled to the sensitive layer film member 68 and are communicatively coupled to the processing device 606 (FIG. 6) via traditional electrical options appreciated by those skilled in the art. For example, by a plurality of wire connectors 78 (for example, as depicted in FIG. 2). Further, each of the plurality of fingers 87 that are interdigitated of the respective comb shaped electrodes 84, 86 are spaced apart from one another. In some embodiments, the spacing between the plurality of fingers 87 or tangs of the comb shaped electrodes 84, 86 of the plurality of electrodes 70 is uniform. In other embodiments, the spacing between the plurality of fingers 87 or tangs of the comb shaped electrodes 84, 86 of the plurality of electrodes 70 is non-uniform.

The diaphragm film assembly 64 is configured to flexibly move between a plurality of positions based on an applied pressure that displaces the diaphragm film assembly 64. In particular, the base attachment layer film member 72, the capacitance layer film member 66, and the sensitive layer film member 68 flexibly move between the plurality of positions in and out of the sensor assembly 46 and the pocket portion 49. This amount of movement, or displacement, of the capacitance layer film member 66 is detected by the sensor assembly 46 via a capacitance change, measured by the sensing capacitor 58, which occurs upon a displacement of the capacitance layer film member 66 relative to the capacitance plate 54b such that as the capacitance layer film member 66 moves or displaces, the space between the capacitance plate 54b and the inner sensor surface 52 of the diaphragm film assembly 64 changes, which changes the capacitance. The measured capacitance change is correlated and used to determine an applied pressure of the fluid 80 that is actually displacing the diaphragm film assembly 64, as discussed in greater detail herein.

Now referring to FIG. 4B, the diaphragm film assembly 64 is schematically depicted in a deflected position. That is, a pressure or a force of the fluid 80 is being applied to the diaphragm film assembly 64 to move or deflect the diaphragm film assembly 64 from the static state, as best shown in FIG. 4A, into the deflected position, as best shown in FIG. 4B. It should be appreciated that the fluid 80 may be a blood flow from a hepatic vein, a portal vein, and the like, which may cause the diaphragm film assembly 64 to move from a static position to a deflected position. As discussed in greater detail herein, the amount of deflection may be determined and correlated to an amount of a measured pressure. Further, as shown, the layers of the diaphragm film assembly 64 deflect while maintaining contact with the pair of legs 74, which may cause the different layers to flex inward towards and may be into the sensor assembly 46. As such, the different layers of the diaphragm film assembly 64 may move from a linear arrangement, as best shown in FIG. 4A, into an arcuate or curvilinear arrangement, as best shown in FIG. 4B.

When a pressure or force of the fluid 80 comes in contact with diaphragm film assembly 64, the base attachment layer film member 72, the capacitance layer film member 66, and the sensitive layer film member 68 each flexibly move or displace from the static state (FIG. 4A) to the deflected position (FIG. 4B). The deflected position may be a plurality of different positions based on the amount of pressure. Further, in the deflected position, the base attachment layer film member 72, the capacitance layer film member 66, and the sensitive layer film member 68 are moved from a generally linear static shape to a curvilinear or arcuate shape. It should be appreciated that, in some embodiments, the pair of legs 74 may remain static and the films of the diaphragm film assembly 64 move, or deflect, with respect to the pair of legs 74. As such, the pair of legs 74 may remain in constant contact with, or coupled, to the different layers of the diaphragm film assembly 64 to maintain the location of the diaphragm film assembly 64 within the pocket portion 49 under a pressure.

That is, in the deflected position, the base attachment layer film member 72, the capacitance layer film member 66, and the sensitive layer film member 68 may move or pivot with respect to the pair of legs 74 such that the base attachment layer film member 72, the capacitance layer film member 66, and the sensitive layer film member 68 remain coupled to the inner surface 77 of the pair of legs 74. In other embodiments, the pair of legs 74 may flex or move under pressure and portions may remain coupled to the sensor end 48b. It should be understood that diaphragm film assembly 64 may flexibly move within the pocket portion 49 of the sensor end 48b.

It should be appreciated that the comb shaped electrodes 84, 86 may allow or permit flexibility in the sensitive layer film member 68 to move, or flex, between the static state and the plurality of deflected positions and to remain in communication. Further, in some embodiments, in the static state, the plurality of electrodes 70 may be adjacent to the sensitive layer film member 68, and, as the diaphragm film assembly 64 moves between the plurality of deflected positions, the plurality of electrodes 70 move with the capacitance layer film member 66 in an axial direction such that the plurality of electrodes 70 remain embedded within the capacitance layer film member 66.

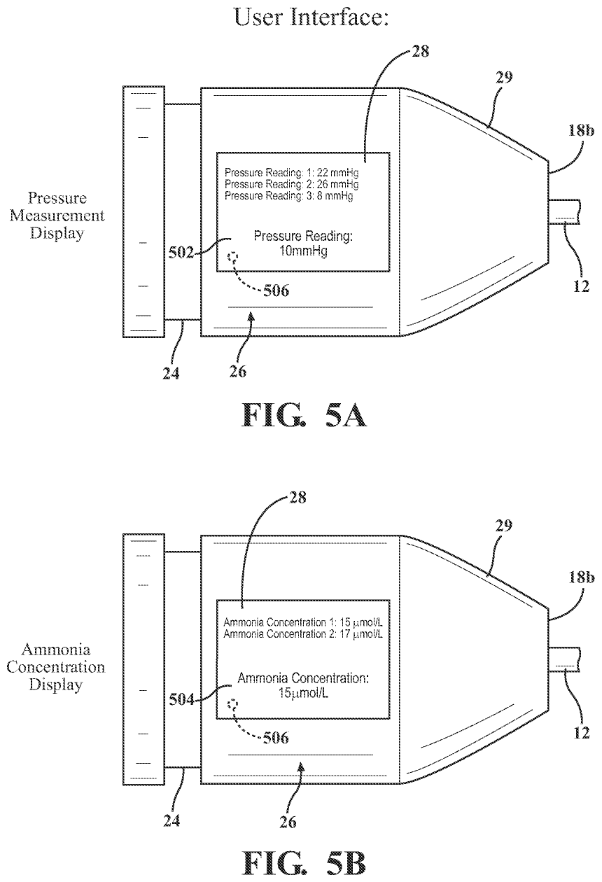

Now referring to FIGS. 5A and 5B, the display device 26 provides a user interface that displays an ammonia concentration, a measured pressure, or both. Both the ammonia concentration and the pressure may be measured in real time from the tip portion 14 (FIG. 1), as discussed in greater detail herein. The display device 26 includes the display 28, which may display a current pressure measurement in millimeters of mercury (mmHg) or Pascal (pa). In some embodiments, and as depicted, the display device 26 may also display previous pressure readings, such as the last three pressure readings 502, though any number of previous pressure reading may be displayed. Further, the display 28 may display a current ammonia concentration measurement in micromoles per liter (μmol/L). The display device 26 may further display one or more previous measurements 504 (e.g., such as two or more previous measurements). As such, the display device 26 may provide a single interface that displays both the pressure and the ammonia concentration in the fluid 80 (FIG. 4B) currently and/or historically at the tip portion 14 (FIG. 1).

The display 28 may include a user input device 506 to capture the current pressure measurement and/or ammonia concentration. In some embodiments, the display 28 may be a touch screen to include the user input device 506. In other embodiments, the user input device 506 may mounted to the handle 16 (e.g., buttons, toggles, switches, and the like) to allow the user to navigate a menu and/or information displayed on the display 28 of the display device 26. It should be appreciated that the user input device 506 may not be positioned on the combination measurement device 10 (FIG. 1) and instead may be initiated by an electronic device, such as a computer, laptop, iPad, and smart mobile phone, and the like. Further, the user input device 506 may have a “zero” ability for capturing direct pressure gradient values. That is, each reading may be stored and the device may be set to “zero” to freshly capture a new direct pressure gradient value(s) and/or differential(s).

Now referring to FIG. 6, an electrical diagram 600 of a combination measuring device 100, according to one or more embodiments is schematically depicted. The electrical diagram 600 generally includes a pressure measurement electrical circuit 602 and an ammonia concentration electrical circuit 604. The processing device 606 is communicatively coupled to both the pressure measurement electrical system and the ammonia concentration electrical system and ultimately to the sensor assembly. Further, the processing device 606 is communicatively coupled to the display device 26 (FIG. 5).

The processing device 606 may be a chip, an integrated circuit, a central processing unit, a mobile electronic device, such as a laptop, mobile device, tablet, and the like.

The pressure measurement electrical circuit 602 may include a voltage source V1, a digital multi-meter DMM, an op amp U1, and a pair of resistors R1, R2, though fewer or more components may be included without departing from the scope of the present disclosure. The op amp U1 and the pair of resistors R1, R2 are configured for signal amplification where a gain is provided by the following equation:

$${v}_{out} = {v}_{i n} * 1 + ( \frac{R 2}{R 1} )$$

Further, the pressure measurement electrical circuit 602 includes a capacitor C2 that may be configured to correct signal attenuation. That is, capacitor C2 may be configured to smooth the pressure measurement signal incoming from capacitor C1, which is the sensed, or detected, capacitance from the sensor assembly 46 (FIG. 2) as a result of an amount of deflection or movement of the capacitance layer film member 66. The digital multi-meter DMM receives the Vout signal from the op-amp U1 and an unaltered signal from the capacitor C1. As such, the digital multi-meter DMM provides an analog to digital capacitance reading that is subsequently processed to a pressure conversion that may be based on a capacitance/pressure curve characterization. It should be appreciated that, in some embodiments, the digital multi-meter DMM is hardware and/or software that is part of the processing device 606, as illustrated in FIG. 6. In other embodiments, the digital multi-meter DMM and the processing device 606 are separate components. In either embodiment, the capacitance/pressure curve characterization may be loaded as software, such as a look-up table or calculations that may be stored within a database of the processing device 606 used for comparison of the digital multi-meter DMM readings 608. Other processing devices may be used in place of a DMM for capacitance reading such as analog comparator circuits equipped with or used in conjunction with analog to digital processing capabilities.

As such, the pressure measurement electrical circuit 602 detects a delta change in pressure at the capacitance layer film member 66 as a pressure output signal. This pressure output signal is displayed on the display device 26 either in real time, where the output is changing, or as an on-demand reading, where the user requests the real time reading for that particular point in time.

Still referring to FIG. 6, the ammonia concentration electrical circuit 604 may include a Wheatstone bridge circuit portion 610 and an op-amp circuit portion 612, though a fewer or greater number of components may be included without departing from the scope of the present disclosure. For example, the ammonia concentration electrical circuit 604 may include a second digital multi-meter DMM. The op-amp portion may include a second voltage source V2, a second op amp U2, and a pair of resistors R3 and R4 that may be configured to detect a change in resistance across the sensitive layer film member 68 via the comb shaped electrodes 84, 86 that include the plurality of fingers 87 and the wire connectors 78.

The Wheatstone bridge circuit portion 610 may be a Wheatstone bridge circuit 614 that includes a third voltage source V3. As illustrated, the Wheatstone bridge circuit 614 may include a plurality of resistors R′. In some embodiments, the plurality of resistors R′ may include three or more resistors, R6, R7, R8 and the resistance measured across the sensitive layer film member 68 via the pair of wire connectors 78 (FIG. 2) forming the last resistor RX. As such, it should be appreciated that the unknown resistance Rx at the sensitive layer film member 68 may be determined by using the known values of resistors R6, R7, and R8. It should be appreciated that the voltage sources V1, V2, and V3 may each be batteries, or other power sources.

As discussed in greater detail herein, each of the comb shaped electrodes 84, 86 of the plurality of electrodes 70 may be of the Wheatstone bridge circuit 614 to output a cumulative resistance indicative of the resistance associated with absorbed ammonia concentration from surrounding fluid at the sensitive layer film member 68. As such, this change in resistance may be correlated to a change in an ammonia concentration by the processing device 606.

The op amp U2 receives the resistance signal from Wheatstone bridge circuit 614. Further, the op-amp circuit portion 612, which may include the op amp U2 and the pair of resistors R3, R4, may be configured for signal amplification where a gain is provided by the following equation:

$${v}_{out} = {v}_{i n} * 1 + ( \frac{R 3}{R 4} )$$

As such, the second digital multi-meter DMM′ receives the Vout signal from the op amp U2 that may include the resistance value from the Wheatstone bridge circuit 614 and that may be amplified through the op-amp circuit portion 612. As such, the change of resistance is determined by the processing device 606 and correlated to an ammonia concentration conversion that may be based on a resistance/ammonia concentration curve characterization. Accordingly, the ammonia concentration electrical circuit 604 may detect a change in resistance of the fluid 80 (FIG. 4B) as an ammonia concentration output signal. This ammonia concentration output signal is displayed on the display device 26 either in real time, where the output is changing, or on-demand, where the user requests the real time reading for that particular point in time.

It should be appreciated that, in some embodiments, the second digital multi-meter DMM′ is hardware and/or software that is part of the processing device 606, as illustrated in FIG. 6. In other embodiments, the second digital multi-meter DMM′ and the processing device 606 are separate components. In either embodiment, the resistance curve characterization may be loaded as software, such as a look-up table or calculations and that may be stored within a database of the processing device 606 used for comparison of the digital multi-meter DMM readings or a database 608 within the processing device 606. Other processing devices may be used in place of a DMM for resistance reading such as analog comparator circuits equipped with or used in conjunction with analog to digital processing capabilities.

It should now be understood that the processing device 606 may be configured to detect a change in a capacitance of the capacitance layer film member 66 output as the pressure output signal that is indicative of the applied pressure currently at the tip portion 14 (FIG. 1) or change in pressure. Further, it should now be understood that the processing device 606 may also be configured to detect a change in a resistance at the sensitive layer film member 68 output as the ammonia concentration output signal in which a change in the resistance is indicative of a direct measurement and/or change in the concentration of ammonia of the fluid 80 (FIG. 4B) at the tip portion 14 (FIG. 1).

Now referring to FIG. 7, a second embodiment of a diaphragm film assembly 702 is schematically depicted. It is understood that the diaphragm film assembly 702 is similar to the diaphragm film assembly 64 with the exceptions of the features described herein. As such, like features will use the same reference numerals with a prefix “7” for the reference numbers. As such, for brevity reasons, these features will not be described again.

The sensitive layer film member 768 and a pair of electrodes 770 of the diaphragm film assembly 702 may be coupled to the distal end 730b of the catheter 712. In particular, the sensitive layer film member 768 and a pair of electrodes 770 may be coupled to the outer surface 732 of the catheter 712 at the distal end 730b. The pair of electrodes 770 are positioned external or internal to the sensitive layer film member 768. The pair of electrodes 770 are interdigitated and arranged with two individually addressable comb shaped electrodes 784, 786. Each of the two individually addressable comb shaped electrodes 784, 786 may include a plurality of fingers 787 or tangs that are spaced apart and arranged to alternate fingers of the plurality of fingers 787 between each of the two individually addressable comb shaped electrodes 784, 786.

In some embodiments, the spacing between the plurality of fingers 787 or tangs of the comb shaped electrodes 784, 786 of the pair of electrodes 770 is uniform. In other embodiments, the spacing between the plurality of fingers 787 or tangs of the comb shaped electrodes 784, 786 of the pair of electrodes 770 is non-uniform. The pair of electrodes 770 are interdigitated with two individually addressable comb shaped electrodes 784, 786. Each of the comb shaped electrodes 784, 786 of the plurality of electrodes 770 are communicatively coupled to the sensitive layer film member 768. In some embodiments, each of the comb shaped electrodes 784, 786 of the plurality of electrodes 770 are also communicatively coupled to the processing device 606 (FIG. 6) via traditional electrical options appreciated by those skilled in the art. For example, by a plurality of positive and negative wires connections 778.

Now referring to FIG. 8, a second embodiment of the tip portion 814 coupled to the catheter 812 is schematically depicted. It is understood that the catheter 812 and the tip portion 814 are similar to the catheter 12 and the tip portion 14 with the exceptions of the features described herein. As such, like features will use the same reference numerals with a prefix “8” for the reference numbers. As such, for brevity reasons, these features will not be described again.

The tip portion 814 may be coupled to and/or integrally (monolithically) formed from the catheter 812. As such, in this embodiment, the tip portion 814 is prohibited from moving between the home position and the deployed position. Instead, the catheter 812 and the tip portion 814 form a single component of the combination measurement device 810.

As illustrated in FIG. 8, the tip portion 814 is coupled to the catheter 812 such that the first end 842a of the tip portion 814 may abut the distal end 830b of the catheter 812. In some embodiments, the first end 842a of the tip portion 814 may be coupled to the distal end 830b of the catheter 812 in a snap fit configuration. In other embodiments, the first end 842a of the tip portion 814 may be coupled to the distal end 830b of the catheter 812 via a fastener, such as a rivet, a hook and loop fastener, a screw, a nut and bolt, epoxy, and the like, such that the first end 842a of the tip portion 814 abuts the distal end 830b of the catheter 812.

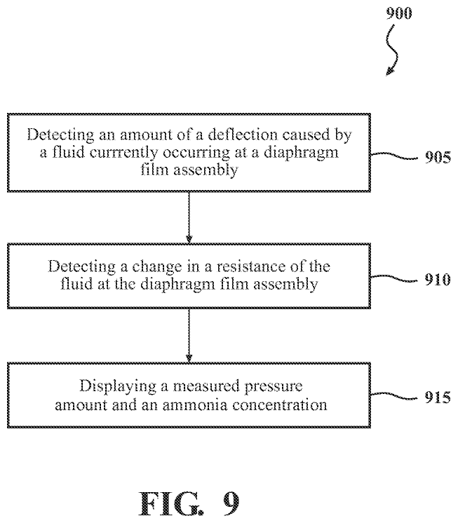

Referring now to FIG. 9, a flow diagram that graphically depicts an illustrative method 900 for determining a pressure and a concentration of ammonia within a fluid is provided. Although the steps associated with the blocks of FIG. 9 will be described as being separate tasks, in other embodiments, the blocks may be combined or omitted. Further, while the steps associated with the blocks of FIG. 9 will described as being performed in a particular order, in other embodiments, the steps may be performed in a different order. Additionally, there may be a greater or fewer number of steps without departing from the scope of the present disclosure.

At block 905, the sensor assembly detects an amount of a deflection currently occurring in the diaphragm film assembly positioned at the tip portion of the catheter. The diaphragm film assembly is communicatively coupled to the sensor assembly. The amount of deflection changes a capacitance in the sensing capacitor. That is, the capacitance sensed in the sensing capacitor is sent to the processing device where the sensed capacitance is compared to the capacitance measured in the reference capacitor and a ratio or amount of the change is determined. As such, the change in capacitance is correlated to a deflection of the capacitor sensing layer film member to determine a pressure of the fluid currently being exerted onto the diaphragm film assembly.

At block 910, a change in a resistance of fluid at diaphragm film assembly is detected by the sensor assembly. In particular, the plurality of electrodes measure the resistance or change in resistance of the sensitive film layer member based on ammonia absorption onto the sensitive film layer member from the fluid. The measured resistance at the plurality of electrodes and/or the sensitive film layer member, along with the known resistance values in the Wheatstone circuit portion are used to determine the actual current measured resistance. As such, the change in the resistance of the sensitive film layer at the diaphragm film assembly is indicative of a measurement or change in the concentration of ammonia.

It should be appreciated that the measurements by the sensing assembly may be direct or indirect, may have increased measuring accuracy compared to conventional measurement techniques, and may take less than 30 seconds per measurement.

At block 915, the amount of the pressure at the diaphragm assembly based on the amount deflection currently occurring and the ammonia concentration based on the change of resistance currently at the diaphragm assembly is displayed on the display portion of the combination measuring device. Further, it should be appreciated that previous measurements may also be displayed for each of the previous pressure measurement readings and the ammonia concentration readings.

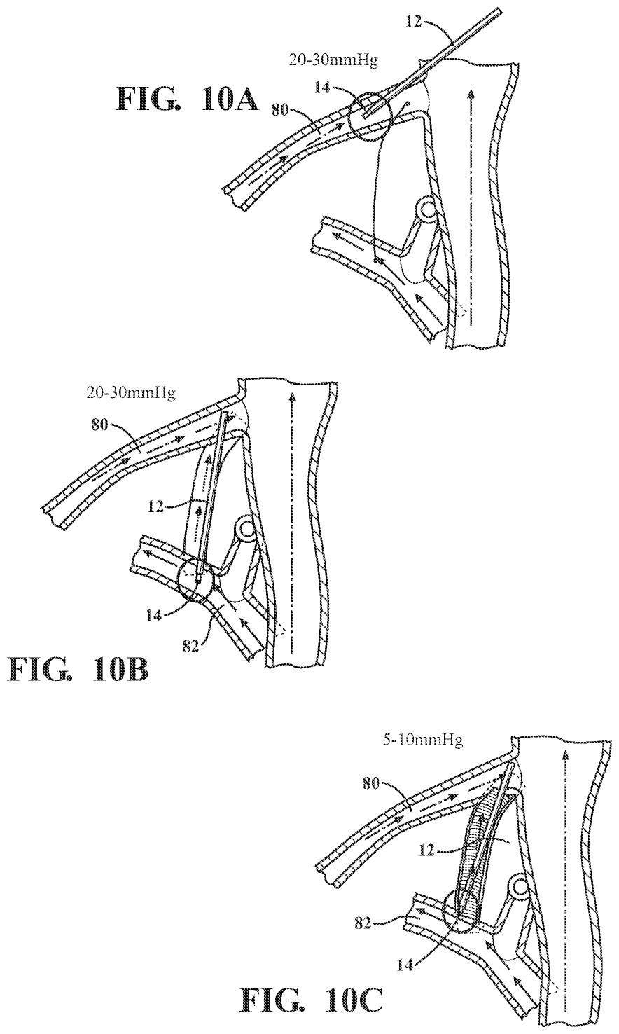

Now referring to FIG. 10A, the tip portion 14 of the combination measurement device 10 is positioned within a hepatic vein 81 pre-TIPS placement. Generally, the tip portion 14 may positioned against a fluid flow such that the fluid flow within the hepatic vein 81 exerts a pressure on the tip portion 14. In this position, the user may be determining whether the pressure measurement is within a range, such as, without limitation, between 20-30 mmHg (2666.45-3999.67 Pa).

Now referring to FIG. 10B, the tip portion 14 of the combination measurement device is positioned within a portal vein 82 pre-TIPS placement. Generally, the tip portion 14 is positioned against the fluid flow. Again, in this position, the user may be determining whether the pressure measurement is within a range, such as, without limitation, between 20-30 mmHg (2666.45-3999.67 Pa).

Now referring to FIG. 10C, the tip portion of the combination measuring device is positioned within the portal vein 82 post-TIPS placement. Generally, the tip portion 14 is positioned against the fluid flow. In this position, the user may be attempting to reduce the pressure gradient between the portal and hepatic veins to a pressure range of interest that typically falls between 5-10 mmHg (666.612-1333.22 Pa). Accordingly, with these measurements, the user may adjust a TIPS placement and/or perform other medical procedures necessary to achieve or maintain the desired pressure ranges.

It should be appreciated that the combination measurement device 10 requires less users than the measurement techniques, provides for a reduced pressure measurement time, provides user confidence in hepatic venous pressure gradient shunt dilation, and eliminates “guess-work” commonly found in the known measuring techniques, thereby reducing risk of over-dilation and associated interventions, and provides for precise measurements.

As such, the embodiments described herein produce desirable results over existing solutions, such as the ability to obtain instantaneous, direct, and accurate pressure measurements and to monitor real-time concentrations of nitrogenous compounds. As such, physicians may anticipate the onset of hepatic encephalopathy (“HE”) such that adjustments to shunt dilation parameters to successfully reach portal decompression while minimizing the risk of HE onset.

Embodiments of the present disclosure may be further described with respect to the following numbered clauses:
1. A measurement device comprising: a processing device; a catheter; a tip portion disposed at a distal end of the catheter, wherein: the tip portion receives the distal end of the catheter; a diaphragm film assembly is coupled to the tip portion at an opposite end that receives the distal end of the catheter, wherein the diaphragm film assembly is configured to flexibly move between a plurality of positions based on an applied pressure that displaces the diaphragm film assembly, and a sensor assembly communicatively coupled to the processing device, the sensor assembly is configured to measure the applied pressure displacing the diaphragm film assembly and the processing device determines a concentration of ammonia currently in a fluid at the tip portion.
2. The measurement device of any preceding clause, wherein the diaphragm film assembly further comprises: a first layer film member, a second layer film member, and a plurality of electrodes positioned between the first layer film member and the second layer film member.
3. The measurement device of any preceding clause, wherein the first layer film member is a PGA Zinc-Oxide Loaded Polymer material and the second layer film member is a capacitive film material.
4. The measurement device of any preceding clause, wherein the first layer film member and the second layer film member flexibly move between the plurality of positions.
5. The measurement device of any preceding clause, wherein a change in a capacitance caused by an amount of movement of the second layer film member is detected by the sensor assembly.
6. The measurement device of any preceding clause, further comprising: the sensor assembly comprises a capacitive micro-electro-mechanical systems type sensor configured to output a first plurality of signals, the processing device is configured to detect a change in a capacitance caused by movement of the second layer film member based on the first plurality of signals, the change in the capacitance caused by movement of the second layer film member is indicative of the applied pressure currently at the tip portion to the processing device.
7. The measurement device of any preceding clause, wherein the sensor assembly is further configured to detect a resistance in the first layer film member, and the processing device is configured to detect a change in the resistance in the first layer film member based on the second plurality of signals received by the processing device indicative of the resistance in the first layer film, and the change in the resistance is indicative of a change in the concentration of ammonia.
8. The measurement device of any preceding clause, wherein each of the plurality of electrodes are interdigitated.
9. The measurement device of any preceding clause, wherein the tip portion further comprises: a cavity extending between the first end portion and the second end portion; and the sensor assembly is positioned within the cavity of the tip portion.
10. The measurement device of any preceding clause, further comprising: a handle, the catheter extending from the handle such that the tip portion is positioned opposite of the handle.
11. The measurement device of any preceding clause, wherein the handle further comprises: a display portion configured to display the applied pressure and the concentration of ammonia in the fluid currently at the tip portion.
12. A combination measurement device comprising: a processing device, a handle; a catheter extending from the handle; and a tip portion disposed at a distal end of the catheter such that the tip portion is positioned opposite of the handle, wherein: the tip portion includes a first end portion, an opposite second end portion, and a cavity extending therebetween, the first end portion receives the distal end of the catheter; a sensor assembly positioned within the cavity of the tip portion; and a diaphragm film assembly coupled to the tip portion and communicatively coupled to the sensor assembly, the diaphragm film assembly configured to flexibly move between a plurality of positions based on an applied pressure that displaces the diaphragm film assembly, wherein the sensor assembly is configured to measure the applied pressure displacing the diaphragm film assembly and the processing device is configured to determine a concentration of ammonia currently in a fluid at the tip portion.
13. The combination measurement device of any preceding clause, wherein the diaphragm film assembly further comprises: a first layer film member; a second layer film member, and a plurality of electrodes positioned between the first layer film member and the second layer film member.
14. The combination measurement device of any preceding clause, wherein the first layer film member is a PGA Zinc-Oxide Loaded Polymer material and the second layer film member is a capacitive film material.
15. The combination measurement device of any preceding clause, wherein a change in capacitance caused by an amount of movement of the second layer film member is detected by the sensor assembly.
16. The combination measurement device of any preceding clause, further comprising: the sensor assembly comprises a capacitive micro-electro-mechanical systems type sensor configured to output a first plurality of signals, the processing device is configured to detect a change in a capacitance caused by movement of the second layer film member based on the first plurality of signals, the change in the capacitance caused by movement of the second layer film member is indicative of the applied pressure currently at the tip portion to the processing device.
17. The combination measurement device of any preceding clause, wherein the sensor assembly is further configured to detect a resistance in the first layer film member, and the processing device is configured to detect a change in the resistance in the first layer film member based on a second plurality of signals received by the processing device indicative of the resistance in the first layer film, the second plurality of signals change in the resistance is indicative of a change in the concentration of ammonia.
18. The combination measurement device of any preceding clause, wherein each of the plurality of electrodes are interdigitated.
19. A method for determining a pressure and a concentration of ammonia within a fluid, the method compromising: detecting, with a sensor assembly, an amount of a deflection currently occurring in a diaphragm film assembly positioned at a tip portion of a catheter, the amount of the deflection changes a capacitance measured by the sensor assembly, the diaphragm film assembly is communicatively coupled to the sensor assembly; detecting, with a processing device, a change in a resistance of diaphragm film assembly; displaying, on a display portion communicatively coupled to the sensor assembly and to the processing device, the amount of the deflection currently occurring and the change in the resistance of diaphragm film assembly, wherein the change in the resistance of diaphragm film assembly is indicative of a change in the concentration of ammonia and an amount of deflection currently occurring in the diaphragm film assembly is indicative of the pressure.
20. The method of any preceding clause, wherein the amount of deflection currently occurring in the diaphragm film assembly caused by the pressure is measured by a change in capacitance of the diaphragm film assembly.

It should now be appreciated that embodiments described herein relate to a combination measurement device that is utilized in conjunction with a covered stent/shunt during a TIPS procedure. The combination measurement device obtains direct pressure measurements at the hepatic and portal veins by using a capacitance change based on a deflection amount of a capacitance film. Further, a concentration of ammonia currently in a fluid at a tip portion is determined by a change in resistance. As such, the change in capacitance correlates to a pressure applied against the tip portion and the change in resistance correlates to an associated concentration of ammonia that bypasses the liver into the hepatic veins. As such, the combination measurement device described herein provides for instantaneous, direct, and accurate pressure measurements and the ability to monitor real-time concentrations of nitrogenous compounds.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

1. A measurement device comprising:
a processing device;
a catheter;
a tip portion disposed at a distal end of the catheter, wherein:
the tip portion receives the distal end of the catheter;
a diaphragm film assembly is coupled to the tip portion at an opposite end that receives the distal end of the catheter,
wherein the diaphragm film assembly is configured to flexibly move between a plurality of positions based on an applied pressure that displaces the diaphragm film assembly, and
a sensor assembly communicatively coupled to the processing device, the sensor assembly is configured to measure the applied pressure displacing the diaphragm film assembly and the processing device determines a concentration of ammonia currently in a fluid at the tip portion.

2. The measurement device of claim 1, wherein the diaphragm film assembly further comprises:
a first layer film member;
a second layer film member, and
a plurality of electrodes positioned between the first layer film member and the second layer film member and communicatively coupled to the second layer film member.

3. The measurement device of claim 2, wherein the first layer film member is a PGA Zinc-Oxide Loaded Polymer material and the second layer film member is a capacitive film material.

4. The measurement device of claim 2, wherein the first layer film member and the second layer film member flexibly move between the plurality of positions.

5. The measurement device of claim 4, wherein a change in a capacitance caused by an amount of movement of the second layer film member is detected by the sensor assembly.

6. The measurement device of claim 5, further comprising:
the sensor assembly comprises a capacitive micro-electro-mechanical systems type sensor configured to output a first plurality of signals, the processing device is configured to detect a change in a capacitance caused by movement of the second layer film member based on the first plurality of signals, the change in the capacitance caused by movement of the second layer film member is indicative of the applied pressure currently at the tip portion to the processing device.

7. The measurement device of claim 6, wherein the sensor assembly is further configured to detect a resistance in the first layer film member, and the processing device is configured to detect a change in the resistance in the first layer film member based on a second plurality of signals received by the processing device indicative of the resistance in the first layer film member, and the change in the resistance is indicative of a change in the concentration of ammonia.

8. The measurement device of claim 2, wherein each of the plurality of electrodes are interdigitated.

9. The measurement device of claim 1, wherein the tip portion further comprises:
a cavity extending between a first end portion and a second end portion; and
the sensor assembly is positioned within the cavity of the tip portion.

10. The measurement device of claim 1, further comprising:
a handle, the catheter extending from the handle such that the tip portion is positioned opposite of the handle.

11. The measurement device of claim 10, wherein the handle further comprises:
a display portion configured to display the applied pressure and the concentration of ammonia in the fluid currently at the tip portion.

12. A combination measurement device comprising:
a processing device,
a handle;
a catheter extending from the handle; and
a tip portion disposed at a distal end of the catheter such that the tip portion is positioned opposite of the handle, wherein:
the tip portion includes a first end portion, an opposite second end portion, and a cavity extending therebetween, the first end portion receives the distal end of the catheter;
a sensor assembly positioned within the cavity of the tip portion; and
a diaphragm film assembly coupled to the tip portion and communicatively coupled to the sensor assembly, the diaphragm film assembly configured to flexibly move between a plurality of positions based on an applied pressure that displaces the diaphragm film assembly,
wherein the sensor assembly is configured to measure the applied pressure displacing the diaphragm film assembly and the processing device is configured to determine a concentration of ammonia currently in a fluid at the tip portion.

13. The combination measurement device of claim 12, wherein the diaphragm film assembly further comprises:
a first layer film member;
a second layer film member, and
a plurality of electrodes positioned between the first layer film member and the second layer film member.

14. The combination measurement device of claim 13, wherein the first layer film member is a PGA Zinc-Oxide Loaded Polymer material and the second layer film member is a capacitive film material.

15. The combination measurement device of claim 13, wherein a change in capacitance caused by an amount of movement of the second layer film member is detected by the sensor assembly.

16. The combination measurement device of claim 13, further comprising:
the sensor assembly comprises a capacitive micro-electro-mechanical systems type sensor configured to output a first plurality of signals, the processing device is configured to detect a change in a capacitance caused by movement of the second layer film member based on the first plurality of signals, the change in the capacitance caused by movement of the second layer film member is indicative of the applied pressure currently at the tip portion to the processing device.

17. The combination measurement device of claim 16, wherein the sensor assembly is further configured to detect a resistance in the first layer film member, and the processing device is configured to detect a change in the resistance in the first layer film member based on a second plurality of signals received by the processing device indicative of the resistance in the first layer film member, the second plurality of signals change in the resistance is indicative of a change in the concentration of ammonia.

18. The combination measurement device of claim 13, wherein each of the plurality of electrodes are interdigitated.

19. A method for determining a pressure and a concentration of ammonia within a fluid, the method compromising:
detecting, with a sensor assembly, an amount of a deflection currently occurring in a diaphragm film assembly positioned at a tip portion of a catheter, the amount of the deflection changes a capacitance measured by the sensor assembly, the diaphragm film assembly is communicatively coupled to the sensor assembly;
detecting, with a processing device, a change in a resistance of diaphragm film assembly;
displaying, on a display portion communicatively coupled to the sensor assembly and to the processing device, the amount of the deflection currently occurring and the change in the resistance of diaphragm film assembly,
wherein the change in the resistance of diaphragm film assembly is indicative of a change in the concentration of ammonia and an amount of deflection currently occurring in the diaphragm film assembly is indicative of the pressure.

20. The method of claim 19, wherein the amount of deflection currently occurring in the diaphragm film assembly caused by the pressure is measured by a change in capacitance of the diaphragm film assembly.
